(19) United States
(12) Patent Application Publication
(10) Pub. No.: US 2019/0196662 A1
(43) Pub. Date: Jun. 27, 2019

(54) GRAPHICAL CONTROL OF GRID VIEWS

(71) Applicant: INTERNATIONAL BUSINESS MACHINES CORPORATION, Armonk, NY (US)

(72) Inventor: Robert John Mitchell, Mississauga (CA)

(21) Appl. No.: 15/851,075

(22) Filed: Dec. 21, 2017

(51) Int. Cl. G06F 3/0481; G06F 17/30; G16H 40/63; G16H 30/40; G06F 3/0484

(52) U.S. Cl. CPC G06F 3/0481; G06F 17/30274; G06F 2203/04803; G16H 30/40; G06F 3/04845; G16H 40/63

(57) ABSTRACT

A computer-implemented process for graphical control of grid views, identifies within a graphical user interface (GUI) comprising a screen filled with an image in a current grid layout, a GUI handle exists in a first position. Responsive to dragging the GUI handle from the first position to a second position, further determining whether a first threshold is met. Responsive to determining that the first threshold is met, generating a new grid layout to fill the screen by dividing the screen into a first number of multiple images, using a single interactive gesture of dragging to change a size, aspect ratio and number of cells, on a fixed size screen.

### BACKGROUND

The present invention relates generally to images viewed in a graphical user interface of a data processing system, and more specifically, to graphical control of the grid views in the graphical user interface of the data processing system.

One image viewing solution generates a lay out of cells within a viewer, during a construction phase, at user-specified positions that sets values for a property defining user-specific options for a static layout. A change of modes from construction mode to a design mode permits a user, using a mouse, to move and resize the cells interactively. Another image viewing solution uses a display pattern that is also user-selectable for displaying thumbnail objects. This user-selectable display pattern includes a number of objects to be displayed, and the display pattern remains constant, even when a window size has been modified. Another image viewing solution provides a capability to analyze images and export the processed images, which may include segmented images, registered images and quantitative features through a network.

Another image viewing solution enables partitioning of an activated image display window into regions, each of which is assigned a set of instructions required for performing one of a set of image manipulation functions. A particular image manipulation function is selected for execution by moving a cursor into a region associated with the particular image manipulation function. Another image viewing solution, in response to a request to scroll a display to present one or more graphical representations above a subset, first determines that a detected input meets predetermined layout-adjustment criteria. A layout of graphical representations above the subset is then configured in which rows above the subset include a different number of graphical representations evenly divisible by a number of columns. The display is then scrolled according to the detected input.

However, there is a need to overcome drawbacks of mode switching, image segmentation, region dependent image manipulation functions and configurations dependent upon being divisible by a number of columns as in the aforementioned techniques.

### SUMMARY

According to an embodiment of the present disclosure, a computer-implemented process for graphical control of grid views identifies, within a graphical user interface (GUI) comprising a screen filled with an image in a current grid layout, a GUI handle exists in a first position. Responsive to dragging the GUI handle from the first position to a second position, a determination is made as to whether a first threshold is met. Responsive to determining that the first threshold is met, a new grid layout is generated to fill the screen by dividing the screen into a first number of multiple images, wherein a single interactive gesture of dragging changes a size, aspect ratio and number of cells, on a fixed size screen.

### BRIEF DESCRIPTION OF THE DRAWINGS

For a more complete understanding of this disclosure, reference is now made to the following brief description, taken in conjunction with the accompanying drawings and detailed description, wherein like reference numerals represent like parts.

FIG. 1 is a block diagram of a network data processing system operable for various embodiments of the disclosure;

FIG. 2 is a block diagram of a data processing system in the network data processing system of FIG. 1 operable for various embodiments of the disclosure;

FIG. 3 is a block diagram representation of components of a graphical grid management system operable for various embodiments of the disclosure;

FIG. 4 is a block diagram representation of a singular graphical grid in accordance with one embodiment of the disclosure;

FIG. 5 is a block diagram representation of a quadrant graphical grid in accordance with one embodiment of the disclosure;

FIG. 6 is a block diagram representation of a multiple graphical grid in accordance with one embodiment of the disclosure;

FIG. 7 is a flowchart of an example process used in the graphical grid management system in accordance with one embodiment of the disclosure; and

FIG. 8 is a flowchart of a generating process in an example process used in the graphical grid management system in accordance with one embodiment of the disclosure.

### DETAILED DESCRIPTION

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

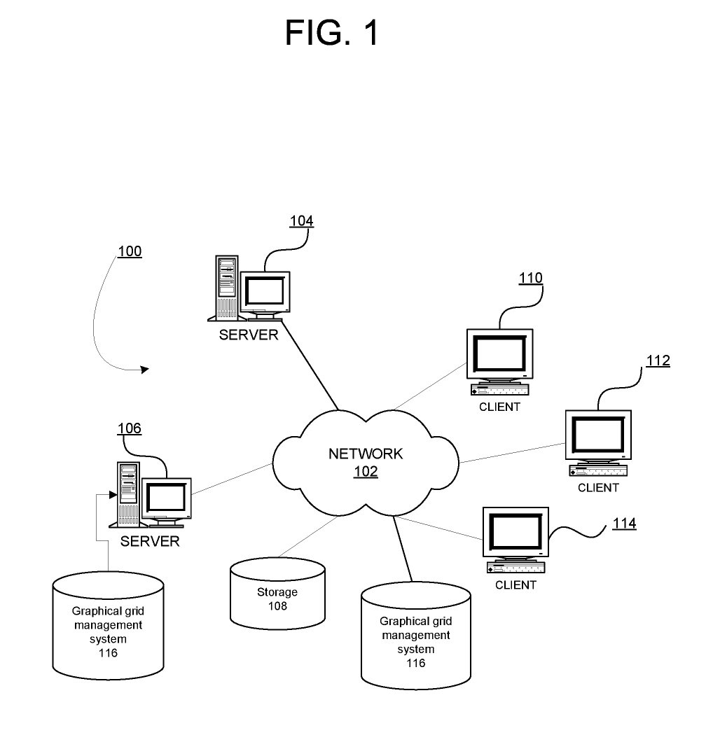

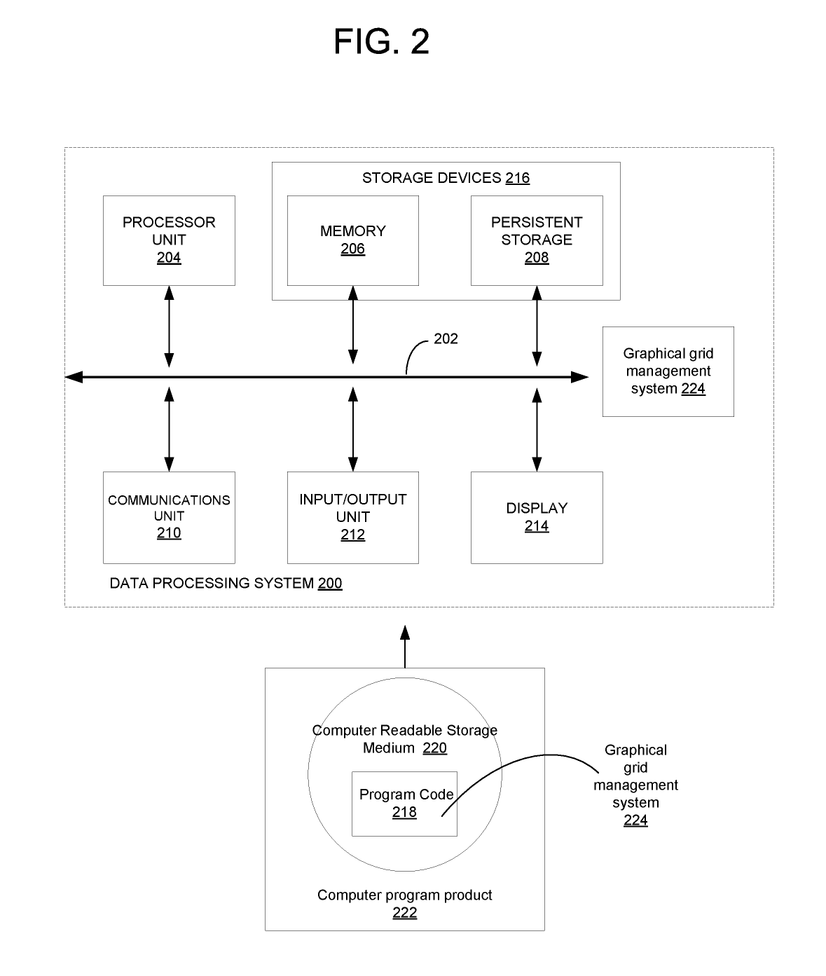

With reference now to the Figures and in particular with reference to FIGS. 1-2, exemplary diagrams of data processing environments are provided in which illustrative embodiments may be implemented. It should be appreciated that FIGS. 1-2 are only exemplary and are not intended to assert or imply any limitation with regard to the environments in which different embodiments may be implemented. Many modifications to the depicted environments may be made.

FIG. 1 depicts a pictorial representation of a network of data processing systems in which illustrative embodiments may be implemented. Network data processing system 100 is a network of computers in which the illustrative embodiments may be implemented. Network data processing system 100 contains network 102, which is the medium used to provide communications links between various devices and computers connected together within network data processing system 100. Network 102 may include connections, such as wire, wireless communication links, or fiber optic cables.

In the depicted example, server 104 and server 106 connect to network 102 along with storage unit 108. In addition, clients 110, 112, and 114 connect to network 102. Clients 110, 112, and 114 may be, for example, personal computers or network computers. In the depicted example, server 104 provides data, such as boot files, operating system images, graphical grid management system 116 and applications to clients 110, 112, and 114. Clients 110, 112, and 114 are clients to server 104 in this example. In addition graphical grid management system 116 may also be directly connected to network 102. Network data processing system 100 may include additional servers, clients, and other devices not shown.

In the depicted example, network data processing system 100 is the Internet with network 102 representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other computer systems that route data and messages. Of course, network data processing system 100 also may be implemented as a number of different types of networks, such as for example, an intranet, a local area network (LAN), or a wide area network (WAN). FIG. 1 is intended as an example, and not as an architectural limitation for the different illustrative embodiments.

With reference to FIG. 2 a block diagram of an exemplary data processing system operable for various embodiments of the disclosure is presented. In this illustrative example, data processing system 200 includes communications fabric 202, which provides communications between processor unit 204, memory 206, persistent storage 208, communications unit 210, input/output (I/O) unit 212, display 214 and graphical grid management system 224.

Processor unit 204 serves to execute instructions for software that may be loaded into memory 206. Processor unit 204 may be a set of one or more processors or may be a multi-processor core, depending on the particular implementation. Further, processor unit 204 may be implemented using one or more heterogeneous processor systems in which a main processor is present with secondary processors on a single chip. As another illustrative example, processor unit 204 may be a symmetric multi-processor system containing multiple processors of the same type.

Memory 206 and persistent storage 208 are examples of storage devices 216. A storage device is any piece of hardware that is capable of storing information, such as, for example without limitation, data, program code in functional form, and/or other suitable information either on a temporary basis and/or a permanent basis. Memory 206, in these examples, may be, for example, a random access memory or any other suitable volatile or non-volatile storage device. Persistent storage 208 may take various forms depending on the particular implementation. For example, persistent storage 208 may contain one or more components or devices. For example, persistent storage 208 may be a hard drive, a flash memory, a rewritable optical disk, a rewritable magnetic tape, or some combination of the above. The media used by persistent storage 208 also may be removable. For example, a removable hard drive may be used for persistent storage 208. In another example, graphical grid management system 224 may also be contained within memory 206 or persistent storage 208.

Communications unit 210, in these examples, provides for communications with other data processing systems or devices. In these examples, communications unit 210 is a network interface card. Communications unit 210 may provide communications through the use of either or both physical and wireless communications links.

Input/output unit 212 allows for input and output of data with other devices that may be connected to data processing system 200. For example, input/output unit 212 may provide a connection for user input through a keyboard, a mouse, and/or some other suitable input device. Further, input/output unit 212 may send output to a printer. Display 214 provides a mechanism to display information to a user.

Instructions for the operating system, applications and/or programs may be located in storage devices 216, which are in communication with processor unit 204 through communications fabric 202. In these illustrative examples the instructions are in a functional form on persistent storage 208. These instructions may be loaded into memory 206 for execution by processor unit 204. The processes of the different embodiments may be performed by processor unit 204 using computer-implemented instructions, which may be located in a memory, such as memory 206.

These instructions are referred to as program code, computer usable program code, or computer readable program code that may be read and executed by a processor in processor unit 204. The program code in the different embodiments may be embodied on different physical or tangible computer readable storage media, such as memory 206 or persistent storage 208.

Program code 218 is located in a functional form on computer readable storage media 220 that is selectively removable and may be loaded onto or transferred to data processing system 200 for execution by processor unit 204. Program code 218 and computer readable storage media 220 form computer program product 222 in these examples. In one example, computer readable storage media 220 may be in a tangible form, such as, for example, an optical or magnetic disc that is inserted or placed into a drive or other device that is part of persistent storage 208 for transfer onto a storage device, such as a hard drive that is part of persistent storage 208. In a tangible form, computer readable storage media 220 also may take the form of a persistent storage, such as a hard drive, a thumb drive, or a flash memory that is connected to data processing system 200. The tangible form of computer readable storage media 220 is also referred to as computer recordable storage media or a computer readable data storage device. In some instances, computer readable storage media 220 may not be removable. In one example, program code 218 contains program code which when executed causes graphical grid management system 224 to be fully functional.

Alternatively, program code 218 may be transferred to data processing system 200 from computer readable storage media 220 through a communications link to communications unit 210 and/or through a connection to input/output unit 212. The communications link and/or the connection may be physical or wireless in the illustrative examples.

In some illustrative embodiments, program code 218 may be downloaded over a network to persistent storage 208 from another device or data processing system for use within data processing system 200. For instance, program code stored in a computer readable data storage device in a server data processing system may be downloaded over a network from the server to data processing system 200. The data processing system providing program code 218 may be a server computer, a client computer, or some other device capable of storing and transmitting program code 218.

Using the example of FIG. 2, in a computer-implemented process for graphical control of grid views processor unit 204 identifies, within a graphical user interface (GUI) comprising a screen of display 214 filled with an image in a current grid layout, a GUI handle exists in a first position. Responsive to dragging the GUI handle from the first position to a second position, processor unit 204 determines whether a first threshold is met. Responsive to determining that the first threshold is met by processor unit 204, a new grid layout is generated by processor unit 204 to fill the screen of display 214 by dividing the screen into a first number of multiple images, wherein a single interactive gesture of dragging changes a size, aspect ratio and number of cells, on a fixed size screen.

The description, which follows, and the embodiments described therein, is provided by way of illustration of an example, or examples, of particular embodiments of the principles of the present invention. These examples are provided for the purposes of explanation, and not limitation, of those principles and of the invention. In the description, which follows, like parts are marked throughout the specification and the drawings with the same respective reference numerals.

The following detailed description of the embodiments of the present invention does not limit the implementation of the present invention to any particular data processing system programming language. The present invention may be implemented in any data processing system programming language provided that the OS (Operating System) provides the facilities that may support the requirements of the embodiments of the present invention. Any limitations presented may be quite likely a result of a particular type of OS, data processing system programming language, or data processing system and may not be a limitation of the embodiment of the present invention.

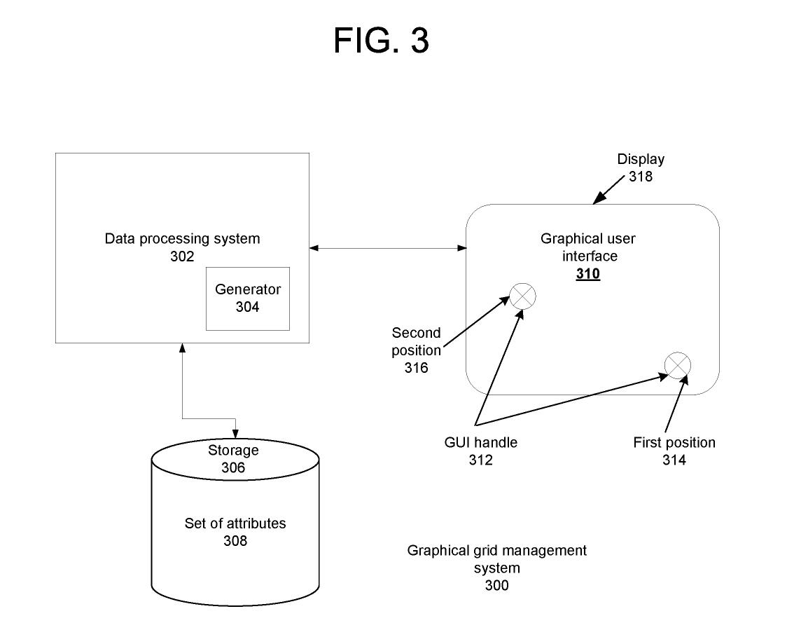

With reference to FIG. 3, a block diagram of components of a graphical grid management system in accordance with one embodiment of the disclosure is presented.

Data processing system 302 is a representation of data processing system 200 and display 318 is representation display 214 of FIG. 2. Generator 304 provides a capability of creating a grid view using input information describing characteristics of display 318 as well as entries in set of attributes 308 maintained with storage 306. Set of attributes 308 contains information used by generator 304 when creating a new layout for presentation on graphical user interface 310. Graphical user interface 310 provides a capability of presenting grid views of images of interest in a screen filled with an image. Graphical user interface 310 also enables a user to interact with graphical grid management system 224 of FIG. 2 at least using GUI handle 312. Typical interaction involves selecting GUI handle 312 in first position 314 and dragging to second position 316.

In the following examples, a medical imaging environment including graphical grid management system 300 of FIG. 3 is presumed. Medical imaging deals with the visualization of body parts including tissues, bones and organs, for a particular use including a clinical diagnosis, a particular treatment based on a finding in an analyzed image and disease monitoring. Various imaging techniques include use of radiology, nuclear medicine and optical imaging. These techniques are typically identified as computed tomography (CT), magnetic resonance imaging (MRI), positron emission tomography (PET), a combination of positron emission tomography and computed tomography (PET-CT), Ultrasound and X-Ray.

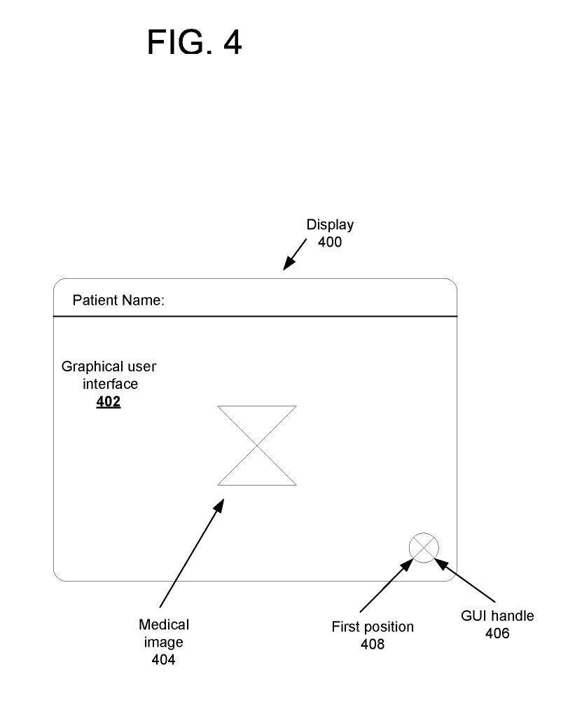

With reference to FIG. 4 a block diagram representation of a singular graphical grid in accordance with one embodiment of the disclosure is presented. In this example embodiment, graphical user interface 402 on display 400 is filled initially with a single instance of medical image 404, and graphical user interface (GUI) handle 406, also known as a hot spot is located in first position 408 in the lower right corner of graphical user interface 402. In this example display 400, graphical user interface 402, medical image 404, graphical user interface (GUI) handle 406, first position 408 are representative of corresponding components including display 318, graphical user interface 310, medical image 404, GUI handle 312, first position 314 of graphical grid management system 300 of FIG. 3.

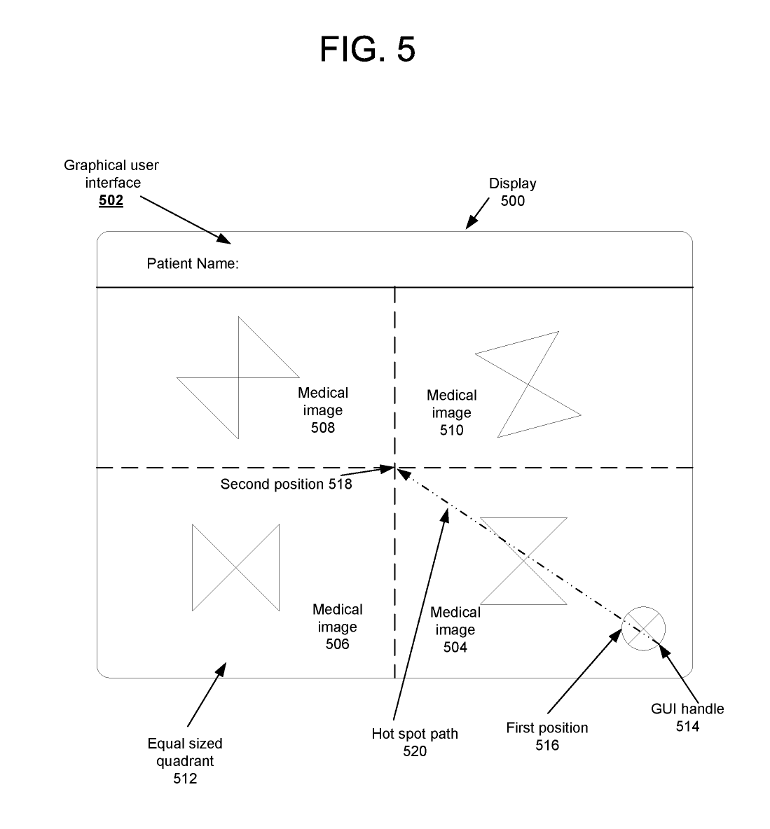

With reference to FIG. 5 a block diagram representation of a quadrant graphical grid in accordance with one embodiment of the disclosure is presented. GUI handle 514 is selected and dragged along an imaginary diagonal, hot spot path 520 across the display screen from an origin of first position 516 of the lower right corner of graphical user interface 502 on display 500, towards the top left corner of display 500. In response to traversing approximately 50% of the distance to the top left corner of display 500, to second position 518, along the imaginary diagonal path, graphical user interface 502 is divided into 4 quadrants, each quadrant being equal size quadrant 512. Each quadrant is dimensioned to be one half of the screen height by one half of the screen width. Each quadrant is then filled with a different one of medical image 504 through 510 received from storage 306 of FIG. 3. In this example display 500, graphical user interface 502, medical image 504 through 510, GUI handle 514, first position 516 are representative of corresponding components including display 318, graphical user interface 310, medical image 404, GUI handle 312, first position 314 of graphical grid management system 300 of FIG. 3.

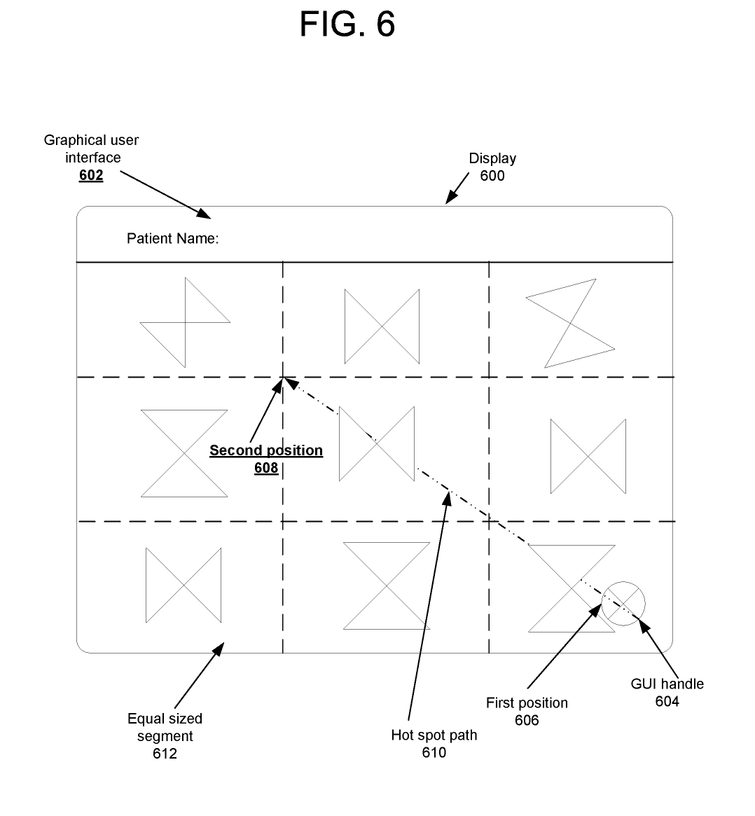

With reference to FIG. 6 a block diagram representation of a multiple graphical grid in accordance with one embodiment of the disclosure is presented.

Continuing the above example, traversal along the imaginary diagonal, hot spot path 610 continues towards the top left corner of display 600, from first position 606 to second position 608, along the imaginary diagonal path, graphical user interface 602 is divided into 9 segments. Each segment being equal size quadrant 612. In this example when GUI handle 604 is approximately ⅓ of the distance from the top right corner of the graphical user interface 602 of display 600 along the imaginary diagonal path, now second position 608, display 600 fills with 9 viewports, represented in a grid of 3 viewports×3 viewports. In a similar manner (not shown), when GUI handle 604 is ¼ of the distance from the top right corner of graphical user interface 602 of display 600 along the imaginary diagonal path, graphical user interface 602 of display 600 fills with 16 viewports, represented in a grid of 4 viewports×4 viewports. In this manner, the display screen is segmented to produce an N×N layout in proportion with the displacement from the origin, first position 606, along the imaginary path.

In another example, not shown but with reference to FIG. 3, commencing with GUI handle 312 positioned in a lower right corner of the display screen, first position 314, moving GUI handle 312 upward in a substantially vertical direction toward the top of graphical user interface 310 of display 318 to second position 316, increases the number of rows in graphical user interface 310 of display 318 but leaves the number of columns unchanged. As a result, graphical user interface 310 of display 318 is divided into 2 sub-screens, splitting graphical user interface 310 horizontally into a top screen and a bottom screen. As GUI handle 312 traverses upward, graphical user interface 310 of display 318 is split horizontally into 3 screens. In a similar manner when GUI handle 312 continues to traverse upward to another second position 316, graphical user interface 310 of display 318 is split horizontally into 4 screens. Moreover, when GUI handle 312 continues to traverse upward to yet another second position 316, graphical user interface 310 of display 318 is further split horizontally into 5 screens.

In another example, not shown but also with reference to FIG. 3, commencing from the bottom right corner of graphical user interface 310 of the display screen and moving GUI handle 312 left horizontally towards the left edge of graphical user interface 310 of display 318, causes graphical user interface 310 to be divided into multiple columns. In this case, the number of columns displayed in graphical user interface 310 is increased but the number of rows is not. As in the previous examples, the number of columns is dependent upon the distance moved from the origin, first position 314 located in the lower right corner. An increment setting predetermines the number of columns generated according to a distance travelled. However, in all examples GUI handle 312 need not be constrained to horizontal, vertical, or diagonal movement. Instead GUI handle 312 is free to range over the entire screen. Furthermore, an origin point may be specified to be a location other than the lower right corner as used in the examples.

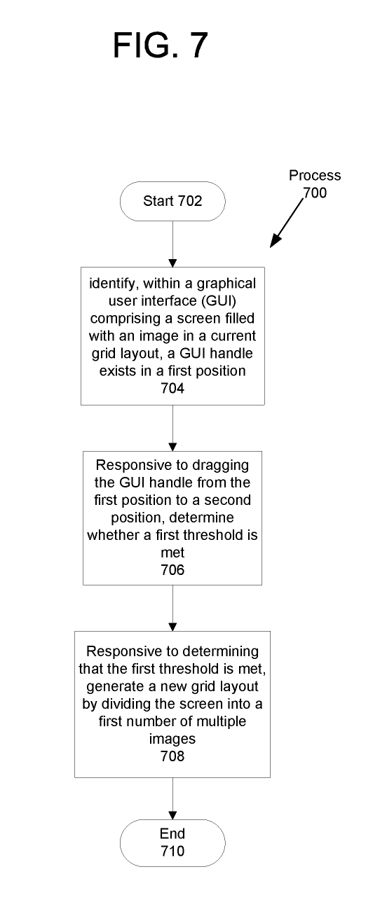

With reference to FIG. 7, a flowchart of an example process using graphical grid management system 300 of FIG. 3 in accordance with one embodiment of the disclosure is presented. Process 700, a computer-implemented process for graphical control of grid views begins (step 702) and identifies, within the graphical user interface (GUI) comprising a screen filled with an image in a current grid layout, a GUI handle exists in a first position (step 704). Responsive to dragging the GUI handle from the first position to a second position, determining whether a first threshold is met (step 706). Responsive to determining that the first threshold is met, generating a new grid layout by dividing the screen into a first number of multiple images, wherein a single interactive gesture of dragging changes a size, aspect ratio and number of cells, on a fixed size screen (step 708) and terminates thereafter (step 710).

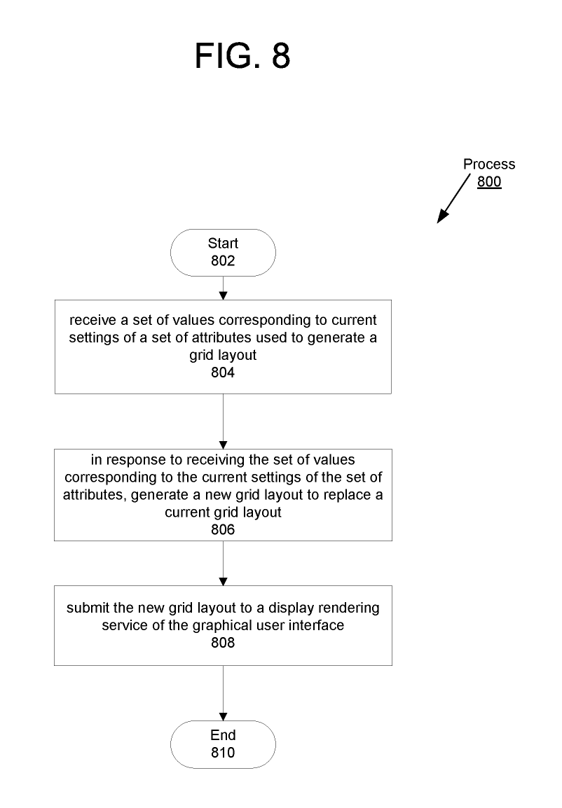

FIG. 8 is flowchart of a generating process in an example of an embodiment, using the graphical grid management system 300 of FIG. 3. Process 800 is an example of a process in which determining that the first threshold is met, further comprises identifying which attributes are in effect to control changing the layout on the display screen.

Process 800 begins (step 802) and receives a set of values corresponding to current settings of a set of attributes used to generate a grid layout (step 804). In response to receiving the set of values corresponding to current settings of the set of attributes process 800 generates a new grid layout to replace a current grid layout (step 806). Process 800 submits the new grid layout to a display rendering service of the graphical user interface, (step 808) and terminates thereafter (step 810).

A determination of a number of cells to produce in a generated grid is a function of a set of attributes including the size of the display area, a type of image being displayed, an increment setting, a first threshold and a vector. The vector identifies the direction and displacement from an origin or starting point. The type of image being displayed identifies a particular property of an image. For example when the image, as originally displayed, is a digital mammogram, there is a need to maintain images in sets of 2 or 4 to present a mediolateral oblique (MLO) view and a craniocaudal (CC) view for each of a left portion and a right portion.

The size of the display area is typically expressed in units of distance or picture elements. An additional attribute specifies an aspect ration used in cell sizing to be one of equivalent size or non-uniform dimensions. The increment setting specifies for each threshold traversed whether a layout is changed from a previous layout by an atomic unit, which maintains a proportional change or more than one unit. For example, when the increment is specified as 2 and the hot spot traverses upward, crossing a threshold of traversal causes the screen to split by a factor of 2, as 1 splits into 2 and 2 screens split into 4. The first threshold contains a value for the displacement trigger point regarding movement of the GUI handle from an origin or first position.

The selection methods described using the graphical grid management system 300 of FIG. 3 thus presented are intuitive, and once used are readily recalled. Typical grid layout tools increase the size of cells when the grid boundary is stretched while adding cells, in the form of rows or columns, is a separate task. In contradistinction the example embodiments describe use of a single interactive gesture that changes the size, aspect ratio and number of cells, on a fixed size screen in a very intuitive way. Using an alternative embodiment, a user can automatically adjust the size of the cells to be of equivalent size. Using another alternative embodiment, the user can automatically cause the cells can be formed with non-uniform dimensions.

The various embodiments thus described enable a user to quickly to specify a number of cells, or viewports that an application displays in a matrix or grid layout. The various embodiments are used when a user desires to quickly choose one of a fixed set of common layouts, for example, 1 row×1 column or 3 rows by 5 columns, because that particular layout enables the user to best compare images of the anatomy including a before view and an after view, a multiple of views of anatomy and a multiple of slices of anatomy.

The example embodiments describe a single interactive gesture that changes the size, aspect ratio and number of cells, on a fixed size screen in a very intuitive way. Free form positioning of N columns and M rows is possible because an embodiment would translate a mouse position, for example the hotspot position, into an optimal N columns and M rows layout. Furthermore, an embodiment has predetermined limits to prevent underflow errors and illogical combinations, for example a maximum of N columns and M rows.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Thus is presented in an illustrative embodiment a computer-implemented process for graphical control of grid views. The computer-implemented method identifies, within a graphical user interface (GUI) comprising a screen filled with an image in a current grid layout, a GUI handle exists in a first position. In response to dragging the GUI handle from the first position to a second position, a determination is made as to whether a first threshold is met. In response to determining that the first threshold is met, a new grid layout is generated to fill the screen by dividing the screen into a first number of multiple images. A single interactive gesture of dragging changes a size, aspect ratio and number of cells, on a fixed size screen.

The flowchart and block diagrams in the figures illustrate the architecture, functionality, and operation of possible implementations of systems, methods, and computer program products according to various embodiments of the present invention. In this regard, each block in the flowchart or block diagrams may represent a module, segment, or portion of code, which comprises one or more executable instructions for implementing a specified logical function. It should also be noted that, in some alternative implementations, the functions noted in the block might occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The invention can take the form of an entirely hardware embodiment, an entirely software embodiment or an embodiment containing both hardware and software elements. In a preferred embodiment, the invention is implemented in software, which includes but is not limited to firmware, resident software, microcode, and other software media that may be recognized by one skilled in the art.

It is important to note that while the present invention has been described in the context of a fully functioning data processing system, those of ordinary skill in the art will appreciate that the processes of the present invention are capable of being distributed in the form of a computer readable data storage device having computer executable instructions stored thereon in a variety of forms. Examples of computer readable data storage devices include recordable-type media, such as a floppy disk, a hard disk drive, a RAM, CD-ROMs, DVD-ROMs. The computer executable instructions may take the form of coded formats that are decoded for actual use in a particular data processing system.

A data processing system suitable for storing and/or executing computer executable instructions comprising program code will include one or more processors coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modems, and Ethernet cards are just a few of the currently available types of network adapters.

What is claimed is:

1. A computer-implemented process for graphical control of grid views, the computer-implemented method comprising:
identifying, within a graphical user interface (GUI) comprising a screen filled with an image in a current grid layout, a GUI handle exists in a first position;
responsive to dragging the GUI handle from the first position to a second position, determining whether a first threshold is met; and
responsive to determining that the first threshold is met, generating a new grid layout to fill the screen by dividing the screen into a first number of multiple images, wherein a single interactive gesture of dragging changes a size, aspect ratio and number of cells, on a fixed size screen.

2. The computer-implemented process of claim 1, wherein responsive to determining that the first threshold is met, further comprises:
receiving a set of values corresponding to current settings of a set of attributes used to generate a grid layout;
generating the new grid layout to replace the current grid layout using the set of values corresponding to current settings of the set of attributes; and
submitting the new grid layout to a rendering service of the graphical user interface.

3. The computer-implemented process of claim 2, wherein the set of values corresponding to current settings of a set of attributes used to generate the grid layout further comprises information representative of body parts including tissues, bones and organs, and an imaging technique including use of radiology, nuclear medicine and optical imaging associated with the image, wherein a techniques is selected from a set consisting of computed tomography (CT), magnetic resonance imaging (MRI), positron emission tomography (PET), a combination of positron emission tomography and computed tomography (PET-CT), Ultrasound and X-Ray.

4. The computer-implemented process of claim 1, wherein the size of the cells is automatically adjusted to one of equivalent size and non-uniform dimensions.

5. The computer-implemented process of claim 2, wherein an increment setting specifies for each threshold traversed that the new grid layout is changed from the current layout by one of an atomic unit, which maintains a proportional change and more than one unit.

6. The computer-implemented process of claim 1, wherein responsive to determining that the first threshold is met, generating the new grid layout by dividing the screen into a first number of multiple images further comprises predetermined limits to prevent underflow errors and illogical combinations.

7. The computer-implemented process of claim 2, wherein the set of attributes further comprises a size of a display area, a type of image being displayed, an increment setting, a first threshold and a vector, wherein the vector identifies a direction and a displacement from an origin and the type of image being displayed identifies a particular property of the image.

8. A data processing system for graphical control of grid views, the data processing system comprising:
a bus;
a memory connected to the bus, wherein the memory contains computer executable instructions;
a processor unit connected to the bus, wherein the processor unit executes the computer executable instructions to direct the data processing system to:
identify, within a graphical user interface (GUI) comprising a screen filled with an image in a current grid layout, a GUI handle exists in a first position;
responsive to dragging the GUI handle from the first position to a second position, determine whether a first threshold is met; and
responsive to determining that the first threshold is met, generate a new grid layout to fill the screen by dividing the screen into a first number of multiple images, wherein a single interactive gesture of dragging changes a size, aspect ratio and number of cells, on a fixed size screen.

9. The data processing system of claim 8, wherein the processor unit executes the computer executable instructions responsive to determining that the first threshold is met, further directs the data processing system to:
receive a set of values corresponding to current settings of a set of attributes used to generate a grid layout;
generate the new grid layout to replace the current grid layout using the set of values corresponding to current settings of the set of attributes; and
submit the new grid layout to a rendering service of the graphical user interface.

10. The data processing system of claim 9, wherein the set of values corresponding to current settings of a set of attributes used to generate the grid layout further comprises information representative of body parts including tissues, bones and organs, and an imaging technique including use of radiology, nuclear medicine and optical imaging associated with the image, wherein a techniques is selected from a set consisting of computed tomography (CT), magnetic resonance imaging (MRI), positron emission tomography (PET), a combination of positron emission tomography and computed tomography (PET-CT), Ultrasound and X-Ray.

11. The data processing system of claim 8, wherein the size of the cells is automatically adjusted to one of equivalent size and non-uniform dimensions.

12. The data processing system of claim 9, wherein an increment setting specifies for each threshold traversed that the new grid layout is changed from the current layout by one of an atomic unit, which maintains a proportional change and more than one unit.

13. The data processing system of claim 8, wherein the processor unit executes the computer executable instructions responsive to determining that the first threshold is met, further directs the data processing system to generate the new grid layout by dividing the screen into a first number of multiple images further comprises predetermined limits to prevent underflow errors and illogical combinations.

14. The data processing system of claim 9, wherein the set of attributes further comprises a size of a display area, a type of image being displayed, an increment setting, a first threshold and a vector, wherein the vector identifies a direction and a displacement from an origin and the type of image being displayed identifies a particular property of the image.

15. A computer program product for graphical control of grid views, the computer program product comprising a computer readable storage medium having program instructions embodied therewith, the program instructions executable by a computer to cause the computer to:
identify, within a graphical user interface (GUI) comprising a screen filled with an image in a current grid layout, a GUI handle exists in a first position;
responsive to dragging the GUI handle from the first position to a second position, determine whether a first threshold is met; and
responsive to determining that the first threshold is met, generate a new grid layout to fill the screen by dividing the screen into a first number of multiple images, wherein a single interactive gesture of dragging changes a size, aspect ratio and number of cells, on a fixed size screen.

16. The computer program product of claim 15, wherein the program instructions executable by a computer responsive to determining that the first threshold is met, further comprise program instructions executable by a computer to cause the computer to:
receive a set of values corresponding to current settings of a set of attributes used to generate a grid layout;
generate the new grid layout to replace the current grid layout using the set of values corresponding to current settings of the set of attributes; and
submit the new grid layout to a rendering service of the graphical user interface.

17. The computer program product of claim 16, wherein the set of values corresponding to current settings of a set of attributes used to generate the grid layout further comprises information representative of body parts including tissues, bones and organs, and an imaging technique including use of radiology, nuclear medicine and optical imaging associated with the image, wherein a techniques is selected from a set consisting of computed tomography (CT), magnetic resonance imaging (MRI), positron emission tomography (PET), a combination of positron emission tomography and computed tomography (PET-CT), Ultrasound and X-Ray.

18. The computer program product of claim 15, wherein the size of the cells is automatically adjusted to one of equivalent size and non-uniform dimensions.

19. The computer program product of claim 16, wherein an increment setting specifies for each threshold traversed that the new grid layout is changed from the current layout by one of an atomic unit, which maintains a proportional change and more than one unit.

20. The computer program product of claim 15, wherein the program instructions executable by a computer responsive to determining that the first threshold is met, further comprise program instructions executable by a computer to cause the computer to:
generate the new grid layout by dividing the screen into a first number of multiple images further comprises predetermined limits to prevent underflow errors and illogical combinations.
